(19) United States
(12) Patent Application Publication
(10) Pub. No.: US 2018/0303677 A1
(43) Pub. Date: Oct. 25, 2018

(54) LACE TENSION-CONTROLLED COMPRESSION SOCK

(71) Applicant: CIRCULATION CONCEPTS, INC., Houston, TX (US)

(72) Inventors: Jessica GRIFFITHS, Pasadena, CA (US); Amy FOX, Fort Collins, CO (US); Nikitha CHERAYIL, Houston, TX (US); Lucas BIZZARO, Houston, TX (US); Crystal LIN, Houston, TX (US)

(21) Appl. No.: 15/958,492

(22) Filed: Apr. 20, 2018

Related U.S. Application Data
(60) Provisional application No. 62/487,854, filed on Apr. 20, 2017.

(51) Int. Cl. A61F 13/08; A41B 11/00

(52) U.S. Cl. CPC A61F 13/085; A41B 11/02; A41B 11/003

(57) ABSTRACT

A lace tension-controlled compression sock that can be easily donned and removed and delivers the necessary amount of pressure to mitigate the effects of chronic venous disorders (CVDs) comprises easily read, graphic pressure indicators for use by the wearer when adjusting the degree of compression.

### CROSS-REFERENCE TO RELATED APPLICATIONS

none

This application claims the benefit of U.S. Provisional Application No. 62/487,854, filed on Apr. 20, 2017, the contents of which are hereby incorporated by reference in their entirety.

### FEDERALLY SPONSORED RESEARCH OR DEVELOPMENT

Not Applicable

### BACKGROUND OF THE INVENTION

### 1. Field of the Invention

The present invention generally relates to compression stockings (socks) for the management of chronic venous disorders (CVD). More particularly, it relates to compression stockings having a user-variable level of compression.

### 2. Description of the Related Art including information disclosed under 37 CFR 1.97 and 1.98

Compression stockings are a specialized hosiery designed to help prevent the occurrence of, and guard against further progression of, venous disorders such as edema, phlebitis and thrombosis. Compression stockings are elastic garments worn around the leg, compressing the limb. This reduces the diameter of distended veins and increases venous blood flow velocity and valve effectiveness. Compression therapy helps decrease venous pressure, prevents venous stasis and impairments of venous walls, and relieves heavy and aching legs.

Knee-high compression stockings are used not only to help increase circulation, but also to help prevent the formation of blood clots in the lower legs. They also aid in the treatment of ulcers of the lower legs.

Unlike traditional dress or athletic stockings and socks, compression stockings use stronger elastics to create significant pressure on the legs, ankles and feet. Compression stockings are tightest at the ankles and gradually become less constrictive toward the knees and thighs. By compressing the surface veins, arteries and muscles, they force circulating blood through narrower channels. As a result, the arterial pressure is increased, which causes more blood to return to the heart and less blood to pool in the feet.

There are two types of compression stockings, gradient and anti-embolism.

In the clinical setting, the applying of the anti-embolism stockings is performed by physicians, nurses and other trained personnel. First, the proper size stocking is determined by measuring the legs. Aseptic technique is not necessary unless the skin is open. The wearer may be placed in the supine position on a bed for fifteen minutes prior to measuring for fit. This allows for venous return and stability before measuring.

Stockings are best applied upon waking before the person gets out of bed, has been sitting or standing and before venous stasis or edema has had a chance to develop.

Fit is critical to the therapeutic effect of compression stockings. A study listed in the American Journal of Nursing in August 2008 showed that compression stockings were incorrectly sized in just under 30% of the cases studied. It found that additional education was needed not only for patients, but also for medical staff.

Gradient compression stockings are designed to remedy impaired “musculovenous pump” performance caused by incompetent leg vein valves. They are woven in such a way that the compression level is highest around the ankle and lessens with increasing distance from the ankle towards the top of the hose.

Physicians will typically recommend these stockings for those who are prone to blood clots, lower limb edema, and blood pooling in the legs and feet from prolonged periods of sitting or inactivity. They are also frequently used to address complications caused by diabetes, lymphedema, thrombosis, cellulitis, and other conditions.

They are worn by those who are ambulatory in most cases, helping calf muscles to perform their pumping action more efficiently to return blood to the heart. In some cases, they are worn by those at increased risk of circulatory problems, such as diabetics, whose legs are prone to excessive swelling. A common indicator for the prescription of such stockings is chronic peripheral venous insufficiency, caused by incompetent perforator veins. Low-pressure compression stockings are available without prescription in most countries, and may be purchased at a pharmacy or medical supply store. Stockings with a higher pressure gradient, say, above 25-30 mmHg, may require a prescription from a doctor.

There are several, crucial, cautionary steps that need to be taken before using compression stockings:

A patient's ankle brachial pressure index (ABPI) must be >1.0 per leg to wear compression stockings, otherwise the stockings may obstruct the patient's arterial flow. The ABI indicates the degree of obstruction of a patient's leg and arm arteries. Any competent doctor or nurse can measure and calculate a patient's ABI.

It is crucial that compression stockings be properly sized. The compression should gradually reduce from the highest compression at the smallest part of the ankle, to a 70% reduction of maximum pressure just below the knee.

Vascular health professionals may use special pads to ensure uniform higher pressure around the circumference of the ankle (to smooth out the irregular cross-sectional profile.) Self-prescription is reasonably safe assuming that the compression gradient is 15-20 mmHg, the ABI (for both legs) is >1.0 and that the stockings fit correctly. “Firm” gradient stockings (20-30 mmHg and 30-40 mmHg) should generally be worn only on medical advice.

Although current research reports mixed results of compression socks on athletic performance, there is anecdotal evidence from athletes that they can benefit from such stockings.

The graduated (gradient, graded) compression stockings and anti-embolism compression stockings come in knee-high and thigh-high length. A systemic review done to compare knee-high and thigh-high graded compression stockings in regards of deep vein thrombosis prevention in medical and surgical patients revealed that there was a 6% risk of developing deep vein thrombosis when wearing knee-high stockings and 4% when wearing thigh-high stockings. It concluded that there was no significant difference in the length of compression stockings when used for deep vein thrombosis prophylaxis. It was suggested that knee-high compression stockings should be the first choice for the deep vein thrombosis prevention in medical and surgical patients. Knee-high stockings are more comfortable, easier to apply, and wearing them increases patients' compliance with treatment. Knee-high stockings are easier to size by limb measurement than thigh-high compression stockings. Thigh-high compression stockings may create a tourniquet effect and cause localized restriction when rolled down. A study of patients treated for post-thrombotic syndrome performed in Italy revealed that redness and itching of the skin was reported in 41% of patients wearing thigh-high and 27% in patients wearing knee-high compression stockings. Consequently, 22% of thigh-high wearers and 14% of knee-high wearers stopped the treatment.

Compression stockings are typically constructed using elastic fibers or rubber. These fibers help compress the limb, aiding in circulation.

Compression stockings are offered in different levels of compression. The unit of measure used to classify the pressure of the stockings is millimeters of mercury (mmHg). They are often sold in a variety of pressure ranges. Over-the-counter support is available in 10-15 or 15-20 mmHg.

Higher pressure stockings require a prescription and a trained fitter. These higher pressures range from 20-30 mmHg to 50+ mmHg.

Other pressure levels used by manufacturers for custom-made, flat-knitted products in the US and Europe range from 18-21 mmHg to >50 mmHg.

It is estimated that 128 million Americans currently suffer from chronic venous disorders (CVD) and require compression for effective blood circulation. Compression socks effectively treat CVDs, but it has been reported that 63% of CVD patients do not wear them as prescribed. Typical complaints are that conventional compression socks are difficult to put on and uncomfortable to wear. The present invention addresses these problems.

### BRIEF SUMMARY OF THE INVENTION

A durable, comfortable, lace tension-controlled compression sock that can be easily donned and delivers the necessary amount of pressure to mitigate the effects of CVD and reduce patient noncompliance comprises easily read, graphic pressure indicators for use by the wearer when adjusting the degree of compression.

### BRIEF DESCRIPTION OF THE SEVERAL VIEWS OF THE DRAWING(S)

FIG. 1 is a perspective view of a first embodiment.

FIG. 2 is a drawing of a compression level-indicating tongue configured for use in the embodiment illustrated in FIG. 1.

FIG. 3 is an exploded view of splines and grommets configured for holding laces in the throat of the embodiment illustrated in FIG. 1.

FIG. 4 is a top plan view of an alternative lace guide for the embodiment illustrated in FIG. 1.

FIG. 4A is a cross-sectional view taken along line A-A in FIG. 4.

FIG. 5 is an exploded view of the lace-tightening device of the embodiment illustrated in FIG. 1.

FIG. 5A is a perspective view of a clutch reel mount configured for use with the lace-tightening device illustrated in FIG. 5.

FIG. 6 is an illustration of an alternative lace-tightening device for use with the embodiment illustrated in FIG. 1.

FIG. 7 is an illustration of a compression sock having an alternative version of compression level-indicating markers.

FIG. 8 is a photograph of the embodiment illustrated in FIG. 1 fitted on the leg of a mannequin.

FIG. 9 is a close-up photograph of the compression level adjustment device of the embodiment shown in FIG. 8.

FIG. 10 is a photograph of an alternative lace guide according to the design illustrated in FIG. 4.

### DETAILED DESCRIPTION OF THE INVENTION

The invention may best be understood by reference to the exemplary embodiments illustrated in the drawing figures wherein the following element numbers are used:

10 lace tension-controlled compression sock

12 clutch reel

14 clutch reel mount

16 lacing

18 zipper

20 tongue

22 internal splines

24 fabric

26 tongue with markers

28 higher compression level marker

30 intermediate compression level marker

32 lower compression level marker

34 holes in spline

36 grommet

38 curved surface

40 central axial bore

42 lace guide

44 lace channel

46 lace tension knob

48 clutch reel-to-holder lock

49 tabs

50 spool

52 clutch reel mount

53 L-shaped slots

54 clutch reel turning accessory

56 clutch reel

58 wrench accessory

60 socket wrench

70 indicating compression sock

72 paired markers

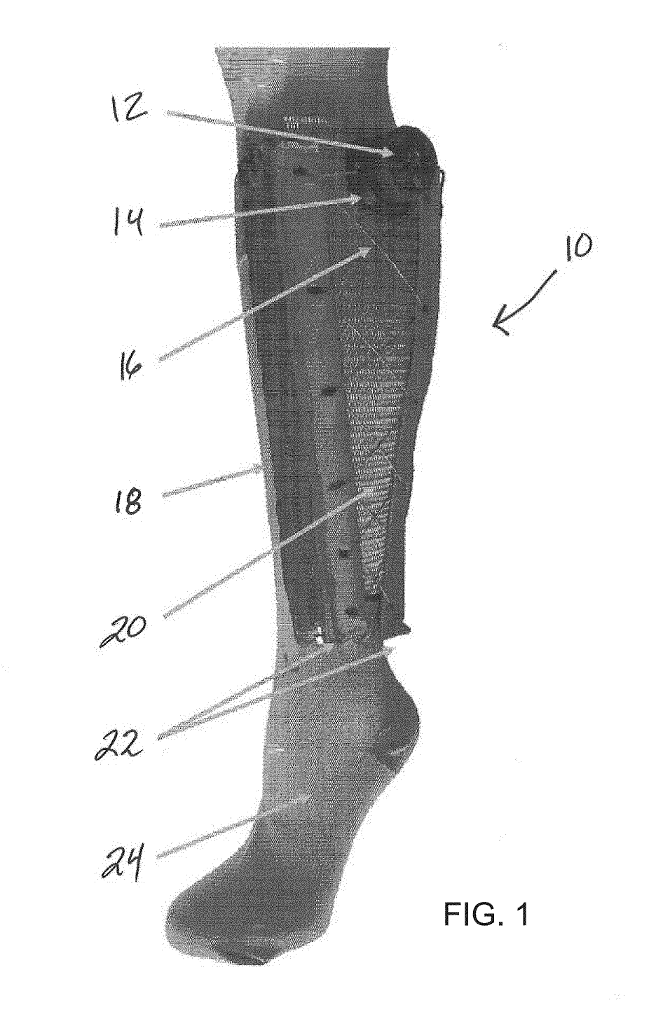

Referring first to FIG. 1, lace tension-controlled compression stocking (or “sock”) 10 may be conventionally formed of knitted stretch fabric 24 which may be a blend of nylon and LYCRA® spandex [Invista North America S.A.R.L. 4123 East 37th Street, North Wichita, Kans. 67220] which provides both elasticity and strength. Unlike conventional compression socks, lace tension-controlled compression sock 10 may have up to about 3½ inches of additional circumference (as compared to conventional compression socks) at its upper end so as to make sock 10 easier for the wearer to don and remove.

In the illustrated embodiment, the adjustment means comprises a laced throat attached to sock fabric 24 at approximately opposing lateral positions on the sock. In keeping with the terminology used in connection with laced footwear, the “throat” is the opening that is opened and closed by the laces and is underlain by the “tongue.” The means of attachment of the adjuster may be of any suitable type. In the illustrated embodiment, zipper(s) 18 are sewn to sock fabric 24 to permit easy detachment and reattachment of clutch reel 12, internal lace splines 22, and lacing 16 as a unit. In this way, sock fabric 24 may be washed and dried separately. In other embodiments, the laced throat is attached directly to sock fabric 24 by, for example, sewing.

The throat of the lace tension-controlled compression sock 10 may be variably closed over tongue 26 by lacing 16 taken up on clutch reel 12. The maximum pressure which may be applied by the device may be constrained by the length of lacing 16.

Lacing 16 may be made of any suitable material. In the illustrated embodiment, lacing 16 is formed of ultra-high-molecular-weight polyethylene (UHMWPE). UHMWPE is a subset of the thermoplastic polyethylene. Also known as high-modulus polyethylene (HMPE), it has extremely long chains with a molecular mass usually between 3.5 and 7.5 million atomic mass units (amu). A longer chain serves to transfer load more effectively to the polymer backbone by strengthening intermolecular interactions. This results in a very tough material, with the highest impact strength of any thermoplastic presently made.

UHMWPE is odorless, tasteless, and nontoxic. It embodies all the characteristics of high-density polyethylene (HDPE) with the added traits of being resistant to concentrated acids and alkalis, as well as numerous organic solvents. It is highly resistant to corrosive chemicals except oxidizing acids; has extremely low moisture absorption and a very low coefficient of friction; is self-lubricating (see boundary lubrication); and is highly resistant to abrasion, in some forms being 15 times more resistant to abrasion than carbon steel. Its coefficient of friction is significantly lower than that of nylon and acetal and is comparable to that of polytetrafluoroethylene (PTFE, “Teflon”), but UHMWPE has better abrasion resistance than PTFE.

UHMWPE is a type of polyolefin. It is made up of extremely long chains of polyethylene, which all align in the same direction. It derives its strength largely from the length of each individual molecule (chain). Van der Waals bonds between the molecules are relatively weak for each atom of overlap between the molecules, but because the molecules are very long, large overlaps can exist, adding up to the ability to carry larger shear forces from molecule to molecule. Each chain is bonded to the others with so many van der Waals bonds that the whole of the inter-molecule strength is high. In this way, large tensile loads are not limited as much by the comparative weakness of each van der Waals bond.

When formed into fibers, the polymer chains can attain a parallel orientation greater than 95% and a level of crystallinity from 39% to 75%.

In an embodiment, lacing 16 is comprised of DYNEEMA® [DSM IP Assets B.V. Het Overloon 1 Heerlen Netherlands NL6411 TE] or SPECTRA® [Vi-Chem Corporation, 55 Cottage Grove Street, S.W. Grand Rapids Mich. 49507] fibers that are formed of lightweight high-strength oriented-strand gel spun through a spinneret. These fibers have yield strengths as high as 2.4 GPa (240 kg/mm² or 350,000 psi) and density as low as 0.97 g/cm³ (for DYNEEMA SK75). High-strength steels have comparable yield strengths, and low-carbon steels have yield strengths much lower (around 0.5 GPa). Since steel has a specific gravity of roughly 7.8, this gives strength-to-weight ratios for these materials in a range from 8 to 15 times higher than steel. Strength-to-weight ratios for DYNEEMA fibers are about 40% higher than that of aramid fibers.

Lacing 16 may be tightened or loosened using clutch reel 12. In the illustrated embodiment, clutch reel 12 is a BOA® lacing system [Boa Technology, Inc. 3459 Ringsby Court, #300 Denver Colo. 80216] various aspects of which are described in U.S. Pat. Nos. 8,091,182, 8,516,662, 7,591,050, 6,289,558, 8,468,657, 7,954,204, 6,424,168, 5,934,599 and 6,202,953 the contents of which are hereby incorporated by reference in their entireties.

Tongue 20 may be made of any suitable material. In the illustrated embodiment, tongue 20 is fabricated of relatively thick (compared to sock fabric 24) [knitted?] nylon. Tongue 20 may float freely within the throat of the device or be attached to fabric 24 by sewing, mechanical fastener(s), adhesive(s), or by hook-and-loop type fasteners. Tongue 20 may be provided with through holes for ventilation and/or may comprise a breathable fabric.

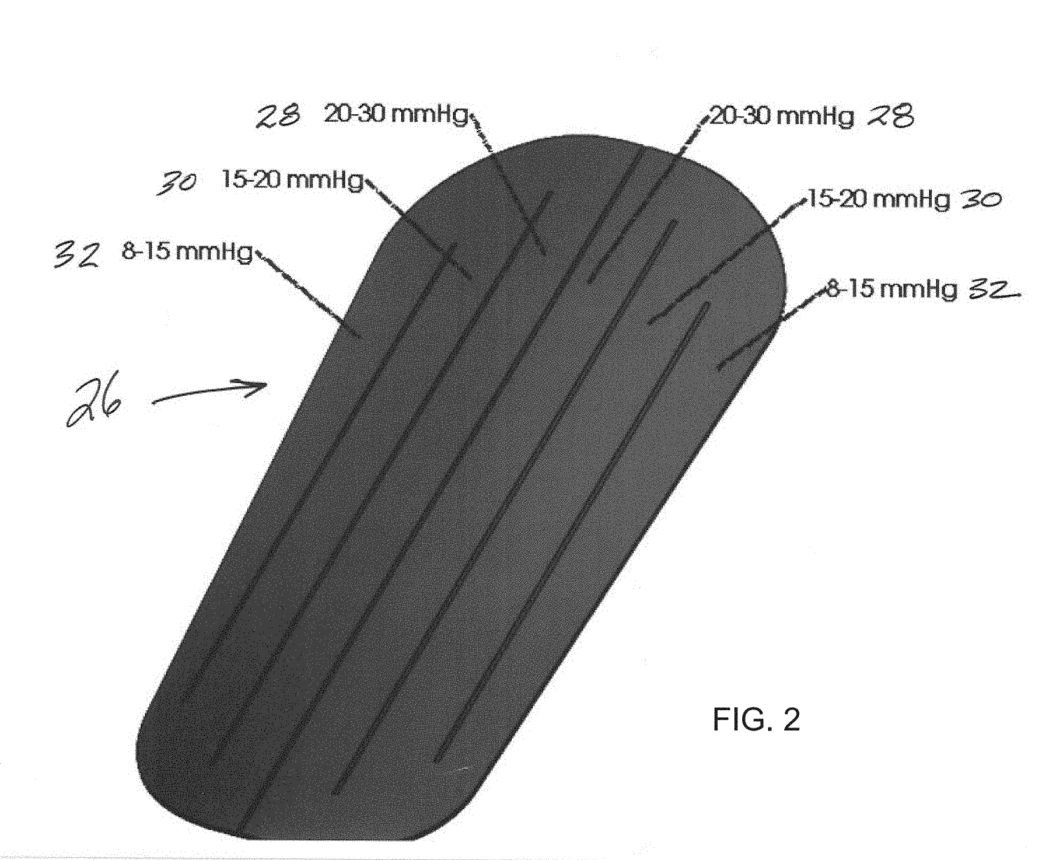

As illustrated in FIG. 2, marker-equipped tongue 26 may have graphical indicators of the width of the opening of the throat—i.e., the exposed portion of tongue 26 between the opposing fabric-covered splines 22 that are pulled towards one another when lacing 16 is tightened using clutch reel 12. In the illustrated embodiment, when the edges of opposing splines 22 are within the areas 32 of tongue 26, lace tension-controlled compression sock 10 is applying between about 8 and about 15 mmHg of pressure; when the edges of opposing splines 22 are within the areas (or “bands”) 32 of tongue 26, lace tension-controlled compression sock 10 is applying between about 15 and about 20 mmHg of pressure; and when the edges of opposing splines 22 are within the areas 38 of tongue 26, lace tension-controlled compression sock 10 is applying between about 20 and about 30 mmHg of pressure. Bands 28, 30 and 32 on tongue 26 may be color-coded or otherwise graphically distinguished.

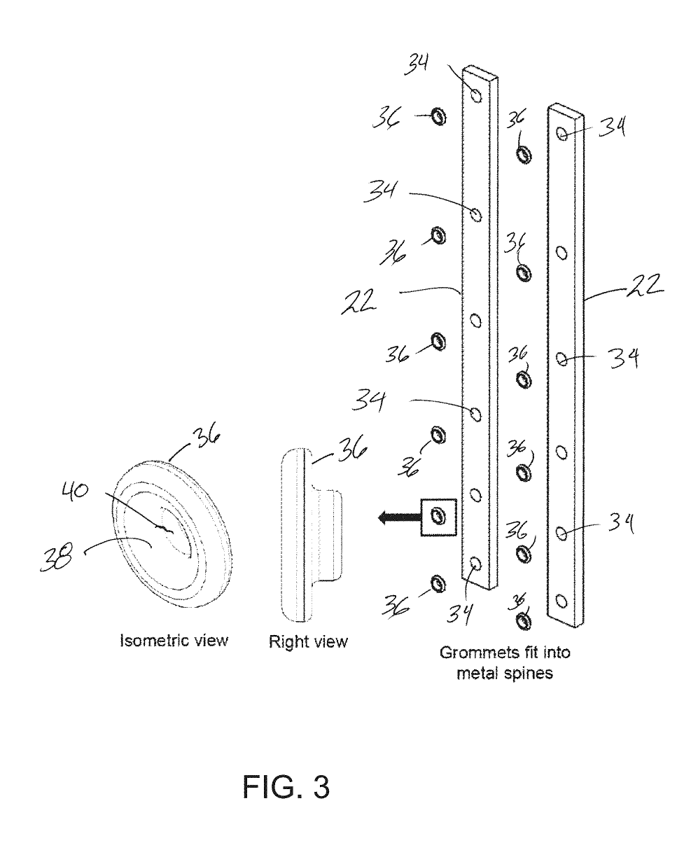

Referring now to FIG. 3, splines 22 may comprise strips of metal or other suitable material and be sewn into pockets attached to zippers 18. Through holes 34 are provided in splines 22 for the passage of lacing 16. The spacing between holes may be varied so as to create a pressure gradient along the length of the throat. Grommets 36 having smooth, curved surface 38 leading to central axial bore 40 may be provided in holes 34 to lessen the likelihood of lacing 16 becoming frayed. Splines 22 may be segmented.

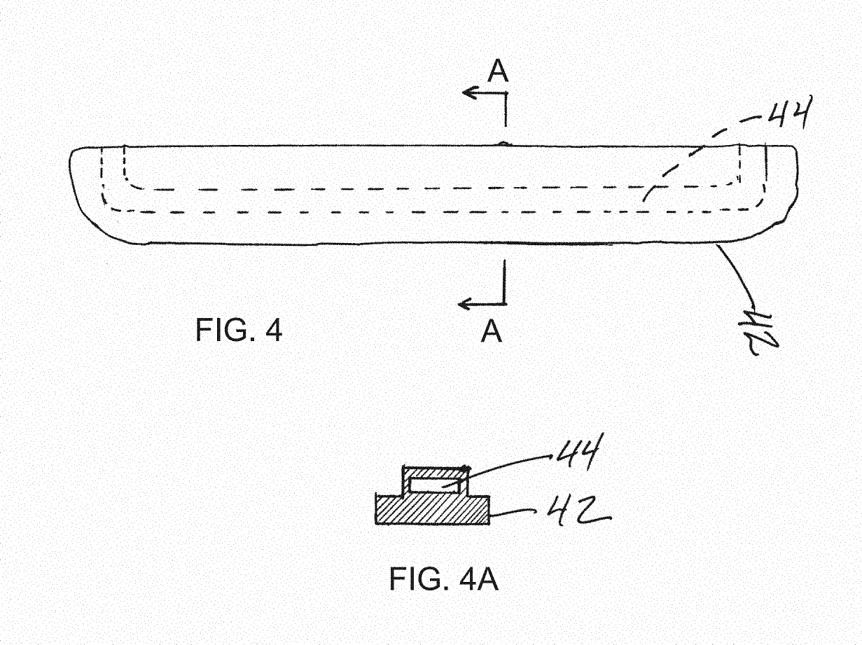

An alternative lacing guide is shown in FIGS. 4 and 4A. Lace guide 42 may have internal channel 44 for the passage of lacing 16. Internal channel 44 may have curved corners to lessen the likelihood of lacing 16 becoming frayed. Lace guide 42 may be sewn into pockets in the same manner as that of splines 22 or may be directly sewn or otherwise attached or adhered to the edges of the throat of the lace tension-controlled device.

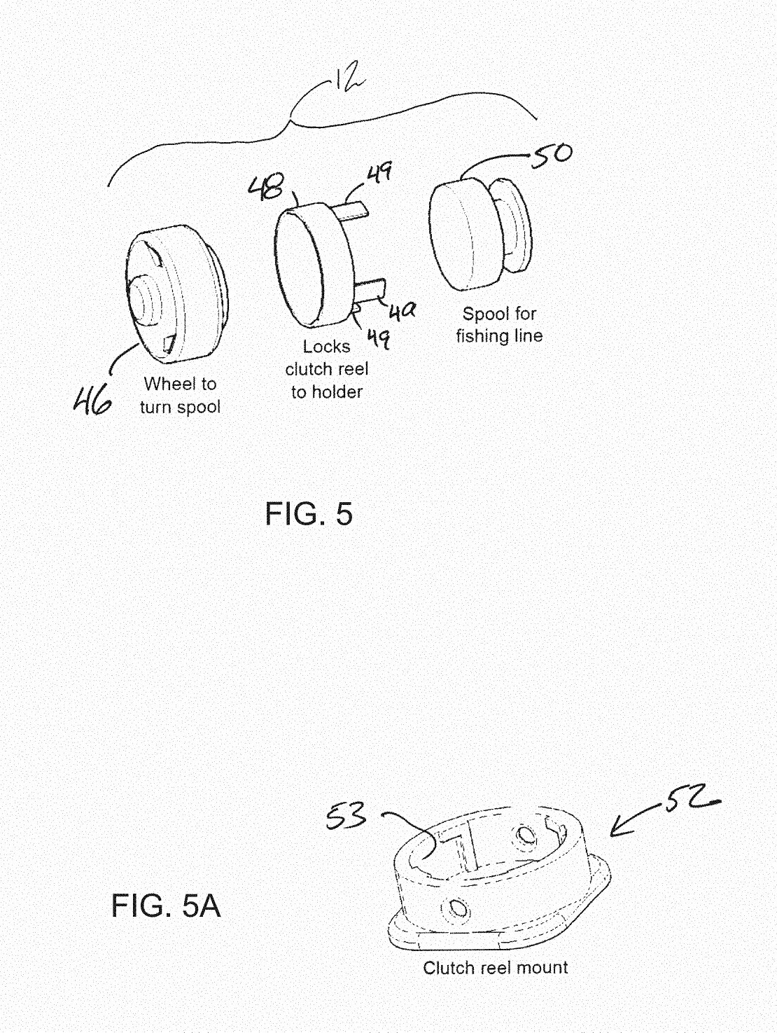

FIG. 5 shows the parts of clutch reel 12 used in the illustrated embodiment. Turning adjustment knob 46 causes lacing 16 to be wound on spool 50. Pulling knob 46 allows spool 50 to freewheel, unwinding lacing 16 and opening the throat of the device. Clutch reel-to-holder 48 is equipped with tabs 49 which engage L-shaped slots 53 in clutch reel mount 52 (see FIG. 5A) to secure the clutch reel 12 on the device. Clutch reel mount 52 may be attached by any suitable means (mechanical or adhesive). In the illustrated embodiment, clutch reel mount 52 is sewn onto tongue 20. Knob 46 may be removable.

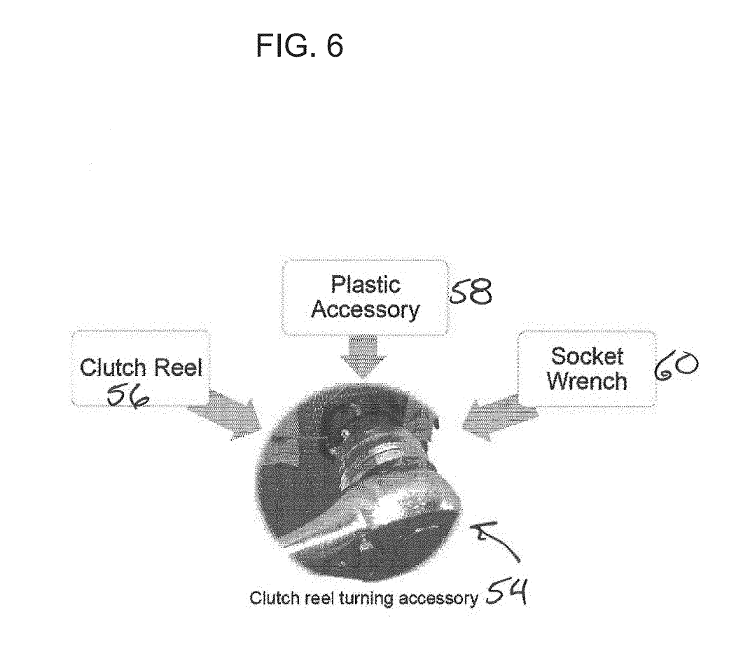

Referring now to FIG. 6, an alternative means for adjusting clutch reel 12 is shown. Accessory 58 may be sized and configured to allow clutch reel 56 to be engaged by socket wrench 60. This allows arm strength rather that hand strength to be used to turn clutch reel 12—an advantage inasmuch as arthritis is a common co-morbidity in CVD patients. In an embodiment, accessory 58 has an outer surface in the shape of a regular hexagon. Socket wrench 60 may be equipped with a correspondingly sized hexagonal socket. Socket wrench 60 may be a torque wrench having a dial indicator or provide a haptic indication of desired torque application. In yet other embodiments, clutch reel 12 may be equipped with a torque-indicating dial or other such readout. A self-adjusting clutch could be used to compensate for changes in the level of edema being experienced by the wearer.

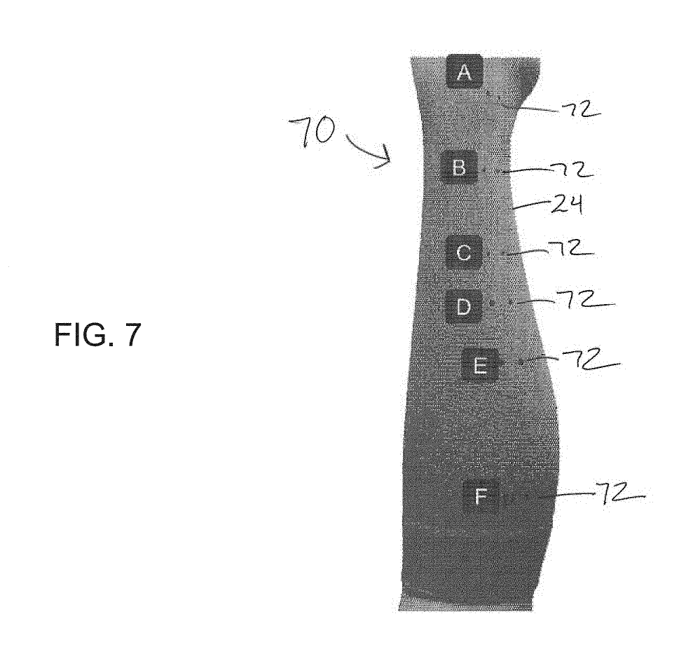

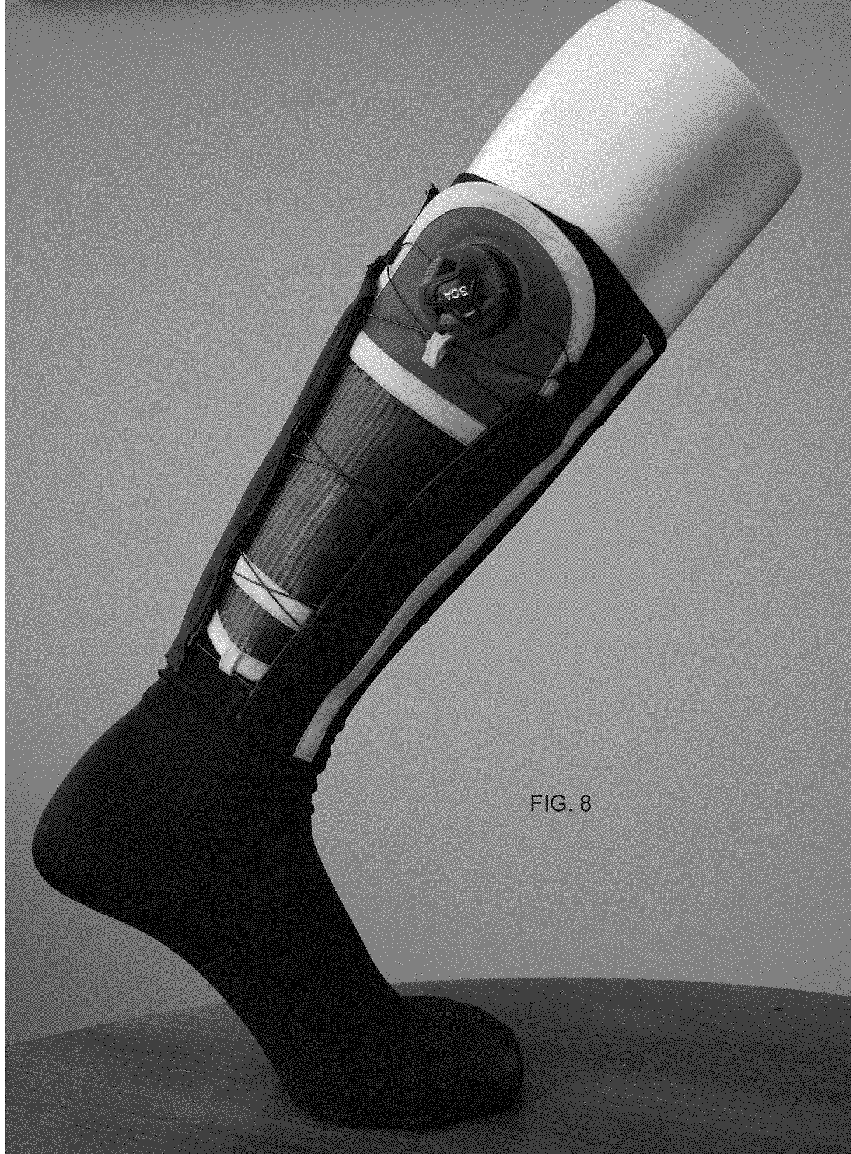

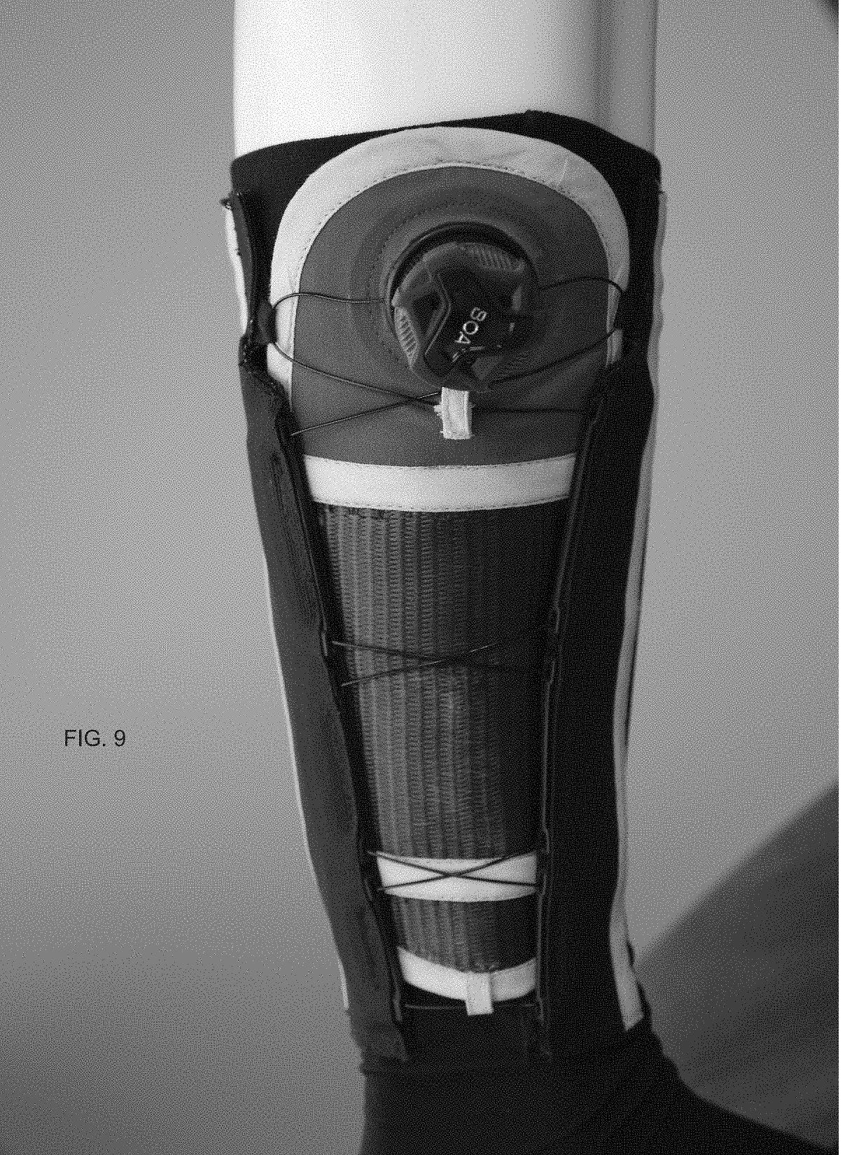

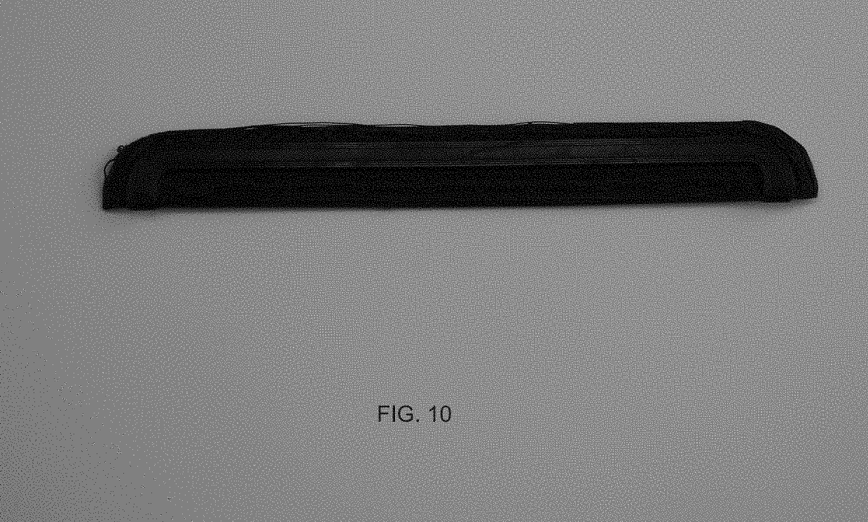

FIG. 7 shows an alternative means for indicating the degree of compression provided by compression sock 70. Indicating compression sock 70 is provided with an array of paired, circumferentially spaced-apart markers 72 on (or in) stretch fabric 24 at locations A through F, inclusive. The more fabric 24 is stretched, the greater will be the distance between the markers of each pair of markers 72. The greater the stretch of fabric 24, the greater the degree of compression applied to the wearer's leg. The distance between markers 72 in each pair of markers may be measured by any conventional means or by spacers specially sized to correspond to a desired degree of compression for each location A-F. Markers 72 may be printed on fabric 24, mechanically or adhesively attached to fabric 24 or woven or knitted into fabric 24. Markers 72 may be provided on the inside surface of the lace tension-controlled compression sock on the side opposite clutch reel 12.

In a test (n=10) of the lace tension-controlled compression sock illustrated in FIG. 1, the average donning time was 22 seconds; the average doffing time was 8 seconds; and the average time required for pressure adjustment was 52 seconds.

As the technology develops, fabric 24 may be provided with pressure sensors and/or temperature sensors. Such sensors may be on-demand devices configured to conserve battery power and may be in wireless data communication with a user's personal electronic device or a health facility's patient monitoring equipment.

The foregoing presents particular embodiments of a system embodying the principles of the invention. Those skilled in the art will be able to devise alternatives and variations which, even if not explicitly disclosed herein, embody those principles and are thus within the scope of the invention. Although particular embodiments of the present invention have been shown and described, they are not intended to limit what this patent covers. One skilled in the art will understand that various changes and modifications may be made without departing from the scope of the present invention as literally and equivalently covered by the following claims.

What is claimed is:

1. A compression sock comprising:
a stretch fabric having one or more pairs of circumferentially spaced-apart markers on or in the fabric, the distance between each marker in a pair of markers becoming greater with increasing compression applied to a limb of a wearer by the compression sock.

2. The compression sock recited in claim 1 wherein the circumferentially spaced-apart markers are woven into the stretch fabric.

3. The compression sock recited in claim 1 wherein the circumferentially spaced-apart markers are knitted into the stretch fabric.

4. The compression sock recited in claim 1 wherein the circumferentially spaced-apart markers are mechanically attached to the stretch fabric.

5. The compression sock recited in claim 1 wherein the circumferentially spaced-apart markers are printed onto the stretch fabric.

6. The compression sock recited in claim 1 wherein the circumferentially spaced-apart markers are in a linear array along a portion of a length of the compression sock.

7. The compression sock recited in claim 1 wherein increasing compression applied to a limb of a wearer by the compression sock is accomplished by increasing a degree of stretch of the stretch fabric.

8. The compression sock recited in claim 7 wherein increasing the degree of stretch of the stretch fabric is accomplished by tightening laces attached to the compression sock.

9. The compression sock recited in claim 8 wherein tightening the laces attached to the compression sock is accomplished by winding the laces on a spool.

10. The compression sock recited in claim 9 wherein the laces and the spool are detachable as a unit from the compression sock.

11. A compression sock comprising:
a stretch fabric;
a laced throat of variable width attached to the stretch fabric;
a tongue within the laced throat;
markers on the on the compression sock indicative of the compression applied by the compression sock when the laced throat is moved from a nominal compression position to a desired compression position.

12. The compression sock recited in claim 11 wherein the width of the throat may be varied by varying a tension applied to the laces.

13. The compression sock recited in claim 12 wherein varying the tension applied to the laces is accomplished by winding the laces on a spool attached to the compression sock.

14. The compression sock recited in claim 13 wherein the tongue, the laces, and the spool are removable from the sock as a unit.

15. The compression sock recited in claim 11 wherein the markers on the compression sock are mechanically attached to the compression sock.

16. The compression sock recited in claim 11 wherein the markers on the compression sock are printed on the compression sock.

17. The compression sock recited in claim 11 wherein the markers on the compression sock are lines on the tongue.

18. The compression sock recited in claim 11 wherein the markers on the tongue are colored bands on the tongue.

19. The compression sock recited in claim 11 wherein increasing compression applied to a limb of a wearer by the compression sock is accomplished by increasing tension applied to the laces.

20. The compression sock recited in claim 11 wherein the spacing of the laces in the laced throat is non-uniform.
